(19) United States
(12) Patent Application Publication
(10) Pub. No.: US 2022/0223242 A1
(43) Pub. Date: Jul. 14, 2022

(54) SYSTEM AND METHOD OF CONTROLLING ACCESS OF A USER'S HEALTH INFORMATION STORED OVER A HEALTH CARE NETWORK

(71) Applicant: Patientory, Inc., Atlanta, GA (US)

(72) Inventor: Chrissa Tanelia McFarlane, Atlanta, GA (US)

(21) Appl. No.: 17/607,215

(22) PCT Filed: Jun. 10, 2019
(86) PCT No.: PCT/US19/36417

§ 371 Date: Oct. 28, 2021

Related U.S. Application Data
(60) Provisional application No. 62/683,513, filed on Jun. 11, 2018.
(60) Provisional application No. 62/683,524, filed on Jun. 11, 2018.
(60) Provisional application No. 62/683,537, filed on Jun. 11, 2018.
(60) Provisional application No. 62/683,556, filed on Jun. 11, 2018.
(60) Provisional application No. 62/683,568, filed on Jun. 11, 2018.

(51) Int. Cl. G16H 10/60; H04L 9/08; H04L 9/30

(52) U.S. Cl. CPC G16H 10/60; H04L 9/30; H04L 9/088

(57) ABSTRACT

A system and a method for controlling access of a user's health information stored over a health care network are described. The method includes providing a Health Information Exchange (HIE) server implemented over a blockchain network. Further, a user device is present in communication with the HIE server. An access of the user's health information may be provided to a third party user based on a user's permission. The user's health information may be stored in at least one of the HIE server and the user device. Further, the user's health information may be updated using a wearable device worn by the user.

### CROSS REFERENCE TO RELATED APPLICATIONS

The present disclosure is related and claims priority under 35 U.S.C. § 1.119(e) to U.S. provisional applications Nos. 62/683,513, entitled SYSTEM AND METHOD FOR MANAGING PAYMENTS FOR ACCESSING PATIENTS INFORMATION; 62/683,524, entitled SYSTEM AND METHOD OF CONTROLLING ACCESS OF A USERS HEALTH INFORMATION STORED OVER A HEALTH CARE NETWORK; 62/683,537, entitled SYSTEM AND METHOD FOR REGULATING A VALUE OF A CRYPTOCURRENCY USED IN A HEALTH CARE NETWORK, 62/683,556, entitled, SYSTEM AND METHOD FOR FACILITATING PAYMENT REQUESTS WITHIN A HEALTH CARE NETWORK, and 62/683568, entitled SYSTEM AND METHOD OF MANAGING ACCESS OF A USERS HEALTH INFORMATION STORED OVER A HEALTH CARE NETWORK, all filed on Jun. 11, 2018, to Chrissa Tanelia McFarlane, the contents of all of which are hereby incorporated by reference in their entirety, for all purposes.

### FIELD OF THE DISCLOSURE

The present disclosure is generally related to a health care network, and more particularly related to a method of controlling access of a user's health information stored over a health care network.

### BACKGROUND

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

To protect important information, utilizing storage on cloud networks is one approach to provide data redundancy. For sensitive information may be stored in an encrypted form. Blockchain leverages both cloud networks and encryption to define storage of all information in a block wise manner The blocks are added to the blockchain in a linear and chronological order. The blockchain helps to store and track data in a secured manner. Currently, the blockchain is used in various fields such as gaming and gambling, diamond industry, real estate, medical industry, or e-voting.

Further, utilization of the blockchain with the various fields involves numerous devices for updating the information. Further, it becomes tedious to maintain a high security when the updated information is accessed by multiple parties through the devices. Therefore, there is a need for an improved system that may provide protection to the seamlessly updated data accessed by the multiple parties.

### SUMMARY

In a first embodiment, a computer-implemented method for accessing a patient's health information includes configuring a health information exchange server in a health care network that includes a blockchain network to communicate with a user device present in communication with the health information exchange server. The computer-implemented method also includes providing an access to the patient's health information to a third party user based on a patient's permission, wherein the patient's health information includes a blockchain string in the blockchain network, and providing the access to the third party user includes providing an encrypted key to decode the blockchain string.

In a second embodiment, a system for accessing a patient's health information includes a memory storing instructions and one or more processors configured to execute the instructions. The instructions cause the system to communicate with a health information exchange server in a health care network that includes a blockchain network and to provide an access to the patient's health information to a third party user based on a patient's permission, wherein the patient's health information includes a blockchain string in the blockchain network, and providing the access to the third party user includes providing an encrypted key to decode the blockchain string. The instructions also cause the system to update the patient's health information using a wearable device worn by a user, wherein the wearable device is present in communication with a user device that has access to the health care network.

In yet other embodiment, a computer-implemented method for accessing a patient's health information includes communicating, from a server in a healthcare network that includes a blockchain network, with a user having a user device, through the healthcare network. The computer-implemented method also includes prompting the user to update the patient's health information with the user device, and receiving a request from a third party user to access the patient's health information. The computer-implemented method also includes providing an access to the patient's health information to the third party user based on a patient's permission, wherein the patient's health information includes a blockchain string in the blockchain network, and providing the access to the third party user includes providing an encrypted key to decode the blockchain string.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 illustrates a network connection diagram 100 of a system 102, according to various embodiments.

FIG. 2A illustrates a method for symmetric encryption of data, according to various embodiments.

FIG. 2B illustrates a method for asymmetric encryption of data, according to various embodiments.

FIG. 3 illustrates a method for hybrid encryption of data, according to various embodiments.

FIG. 4 illustrates a system for storing and accessing data in a health care network, according to various embodiments.

FIG. 5 illustrates a system for storing and accessing data in a health care network implemented, for example, over a blockchain network, according to various embodiments.

FIG. 6A illustrates an example of a table showing various example types of information stored in a patient blockchain database, according to various embodiments.

FIG. 6B illustrates an example of a table showing various example types of information stored in an authorization database, according to various embodiments.

FIG. 6C illustrates an example of a table showing various example types of information stored in a key database, according to various embodiments.

FIG. 6D illustrates an example of a table showing various types of information stored in medical records database, according to various embodiments.

FIG. 7 illustrates a flowchart showing an example method that can be carried out by a medical selection module, according to various embodiments.

FIG. 8 illustrates a flowchart showing an example method that can be performed by a system control module, according to various embodiments.

FIG. 9 illustrates a flowchart showing an example method that can be performed by a monitor and report module, according to various embodiments.

FIG. 10 illustrates a flowchart showing an example method that can be performed by a secure module, according to various embodiments.

FIG. 11 is a block diagram that illustrates a computer system used to perform at least some of the steps and methods in accordance with various embodiments.

### DETAILED DESCRIPTION

Some embodiments of this disclosure, illustrating all its features, will now be discussed in detail. The words “comprising,” “having,” “containing,” and “including,” and other forms thereof, are intended to be open ended in that an item or items following any one of these words is not meant to be an exhaustive listing of such item or items, or meant to be limited to only the listed item or items.

It should also be noted that as used herein and in the appended claims, the singular forms “a,” “an,” and “the” include plural references unless the context clearly dictates otherwise. Although any systems and methods similar or equivalent to those described herein can be used in the practice or testing of embodiments of the present disclosure, particular embodiments of the systems and methods are now described.

Current systems and methods for storing and managing transfer of health information between multiple parties in the healthcare system are often centralized structures subject to hacking, and yet mired in strict security regulations and onerous overhead costs. This state of affairs leads to a lack of efficient and transparent information exchange, to the ultimate detriment of patients and physicians Embodiments as disclosed herein resolve the above technical problem arising in the realm of healthcare data management by implementing a blockchain infrastructure to minimize security breaches and facilitate coordination between multiple entities and organizations, thus improving the health outcomes for patients.

In some embodiments, a blockchain infrastructure as disclosed herein allows the care providers to avoid medication errors, thus reducing the need for duplicate testing. Further, blockchain technology as disclosed herein effectively tracks and timestamps activities related to health information data. Thus, some embodiments provide a robust audit trail that ensures access to all interested and authorized parties to an updated version of a medical record.

Furthermore, some embodiments a blockchain network as disclosed herein includes smart contracts configured with universal parameters. Accordingly, patients become the primary intermediaries for sending and receiving health information. Records stored in a blockchain network as disclosed herein are robust to tampering or error, and stored across multiple participating users (e.g., the entire blockchain network). Accordingly, recovery contingencies are unnecessary. Moreover, the transparency of a blockchain network as disclosed herein substantially reduces the number of data exchange integration points and the need for tedious reporting activities.

In some embodiments, a mobile application installed in client devices allow users to interact with the blockchain network and access features such as messaging, and access updated and accurate health information. Further, some embodiments provide tracking applications and other activity trackers to enable doctors, care providers and other parties in the blockchain network to communicate on a single, easy to use platform. Furthermore, in some embodiments, artificial intelligence, machine learning, neural networks, and other nonlinear algorithms are incorporated to store and manage data in the blockchain network.

Some embodiments provide the ability for patients and other users of the blockchain network to access tokens from an external blockchain to convert into a supported cryptocurrency for access and use of storage features.

Embodiments of the present disclosure will be described more fully hereinafter with reference to the accompanying drawings in which like numerals may represent like elements throughout the several figures, and in which various example embodiments are shown. Embodiments may, however, be embodied in many different forms and should not be construed as limited to the embodiments set forth herein. The examples set forth herein are non-limiting examples and are merely examples among other possible examples.

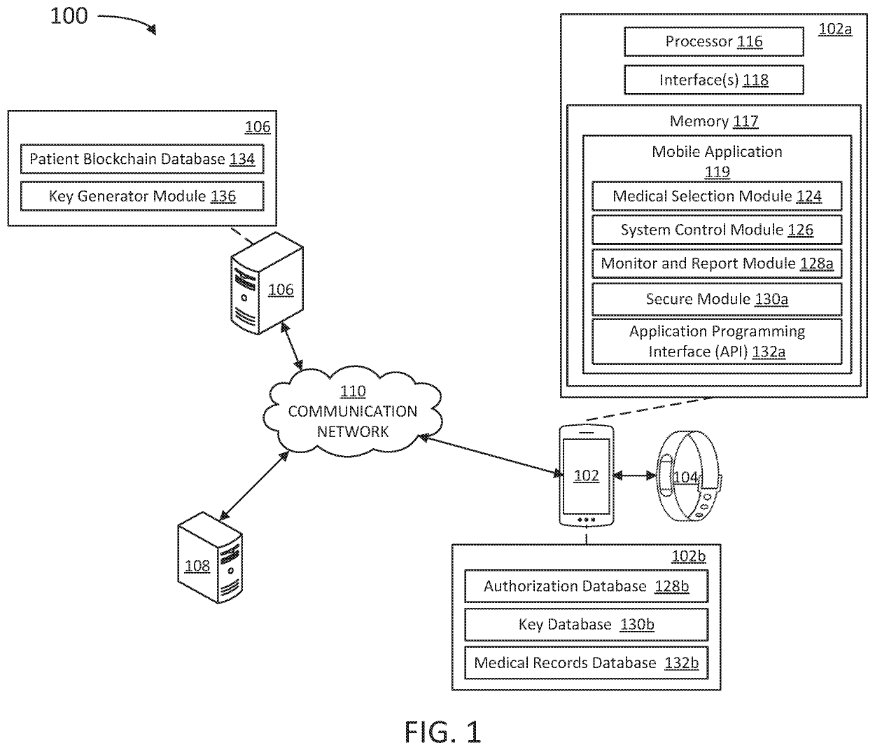

FIG. 1 illustrates a network connection diagram 100 of a system of controlling access of a user's health information stored over a health care network. The system may include a user device 102. The user device 102 may be connected with a wearable device 104. Further, the user device 102 may be connected with a Health Information Exchange (HIE) server 106 and a third party network server 108, through a communication network 110.

The user device 102 may be in communication with the wearable device 104. The wearable device 104 may be used to collect and track user's health information. The user's health information may include one or more parameters such as, but not limited to, blood pressure, heart rate, or number of steps moved per day. The wearable device 104 may be worn by the user. Further, the wearable device 104 may have small motion sensors to capture photos and synchronize with the user device 102. The wearable device 104 may be smartwatches or fitness tracking bands. In accordance with various embodiments, the wearable device 104 may be connected directly to the communication network 110.

The communication network 110 may be a wired and/or a wireless network. The communication network 110, if wireless, may be implemented using communication techniques such as Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques known in the art.

The user device 102 may include a group of components 102a for controlling access of a user's health information stored over a health care network. The group of components 102a may include a processor 116, interface(s) 118, and a memory 117. The memory 117 may include a mobile application 119. The mobile application 119 may include a medical selection module 124, a system control module 126, a monitor and report module 128a, a secure module 130a, and an application programming interface (API) 132.

The processor 116 may execute an algorithm stored in the memory 117 for controlling access of a user's health information stored over a health care network. The processor 116 may also be configured to decode and execute any instructions received from one or more other electronic devices or server(s). The processor 116 may include one or more general purpose processors (e.g., microprocessors) and/or one or more special purpose processors (e.g., digital signal processors (DSPs) or System On Chip (SOC), Field Programmable Gate Array (FPGAs), or Application-Specific Integrated Circuits (ASICs)). The processor 116 may be configured to execute one or more computer-readable program instructions, such as program instructions to carry out any of the functions described in this description.

The interface(s) 118 may help an operator to interact with the user device 102. The interface(s) 118 may either accept inputs from users or provide outputs to the users, or may perform both the actions. In one case, a user may interact with the interface(s) 118 using one or more user-interactive objects and devices. The user-interactive objects and devices may include, for example, user input buttons, switches, knobs, levers, keys, trackballs, touchpads, cameras, microphones, motion sensors, heat sensors, inertial sensors, touch sensors, or a combination of the above. Further, the interface(s) 118 may either be implemented, for example, as a Command Line Interface (CLI), a Graphical User Interface (GUI), a voice interface, or a web-based user-interface.

The memory 117 may include, but is not limited to, fixed (hard) drives, magnetic tape, floppy diskettes, optical disks, Compact Disc Read-Only Memories (CD-ROMs), and magneto-optical disks, semiconductor memories, such as ROMs, Random Access Memories (RAMs), Programmable Read-Only Memories (PROMs), Erasable PROMs (EPROMs), Electrically Erasable PROMs (EEPROMs), flash memory, magnetic or optical cards, or other type of media/machine-readable medium suitable for storing electronic instructions. The memory 117 may include modules implemented as a program.

In accordance with various embodiments, several users may interact via the user device 102. Although a single user device has been illustrated for simplicity, several user devices could similarly be connected to the communication network 110. Further, each of the user devices may have a device ID. In various embodiments, the device ID may be a unique identification code such as an (International Mobile Equipment Identity) IMEI code or a product serial number. It should be noted that a user may use a single user device or multiple user devices. Further, multiple users may use a single user device or multiple user devices. Further, the one or more users may receive and/or provide healthcare related products and services. The one or more users, for example, may include patients, family and friends of the patients, hospitals, physicians, nurses, specialists, pharmacies, medical laboratories, testing centers, insurance companies, or Emergency Medical Technician (EMT) services.

The user device 102 may be a stationary device, a portable device, or a device accessed remotely. The user device 102 may be, but not limited to, a computer, a laptop, a tablet, a mobile phone, a smartphone, or a smart watch. In accordance with various embodiments, the user device 102 may include an imaging device that may be configured to capture a visual graphical element. The visual graphical element may include, but not limited to, a barcode, text, a picture, or any other forms of graphical authentication indicia. In various embodiments, the barcode may be one-dimensional or two-dimensional. Further, the imaging device may include a hardware and/or software element. In various embodiments, the imaging device may be a hardware camera sensor that may be operably coupled to the user device 102. In various embodiments, the hardware camera sensor may be embedded in the user device 102. In accordance with various embodiments, the imaging device may be located external to the user device 102. In accordance with various embodiments, the imaging device may be connected to the user device 102 wirelessly or via a cable. It should be noted that image data of the visual graphical element may be transmitted to the user device 102 via the communication network 110.

In accordance with various embodiments, the imaging device may be controlled by applications and/or software(s) configured to scan a visual graphical code. In various embodiments, a camera may be configured to scan a QR code. Further, in various embodiments, the applications and/or software(s) may be configured to activate the camera present in the user device 102 to scan the QR code. In various embodiments, the camera may be controlled by a processor natively embedded in the user device 102. In various embodiments, the imaging device may include a screen capturing software (for example, screenshot) that may be configured to capture and/or scan the QR code on a screen of the user device 102.

In accordance with various embodiments, the user device 102 may include a group of databases 102b. In various embodiments, the group of databases 102b may be implemented over a blockchain network, and may be present as different databases installed at different locations. In various embodiments, the group of databases 102b may include an authorization database 128b, a key database 130b, and medical records database 132b. In various embodiments. the group of databases 102b may be configured to store data belonging to different users and data required for functioning of the user device 102 and the HIE server 106. Different databases are used in various embodiments disclosed herein; however, a single database may also be used for storing the data in various embodiments. Usage of the different databases may also allow segregated storage of different data and may thus reduce time to access required data. In various embodiments, the data may be encrypted, time-dependent, piece-wise, and may be present as subsets of data belonging to each user. For example, in various embodiments, the data may represent the results of one medical test in a series of multiple medical tests.

In accordance with various embodiments, the group of databases 102b may operate collectively or individually. Further, the group of databases 102b may store data as tables or charts. Further, the group of databases 102b may be configured to store data required or processed by the HIE server 106 implemented over a blockchain network (such as a PTOYNet blockchain network or a PTOYNet EthereumTM Blockchain network). The data may include, but not limited to, a patient medical history, medications, prescriptions, immunizations, test results, allergies, insurance provider, or billing information. Further, the data may be time-dependent and piece-wise. Further, the data may represent a subset of data for each patient. In an example, the data may represent results of a medical test in a series of multiple medical tests. Further, the data may be securely stored. In various embodiments, the data may be encrypted.

In accordance with various embodiments, information stored in the group of databases 102b may be accessed based on users' identities and/or the users' authorities. The users' identities may be verified in one or more ways such as, but not limited to, bio-authentication, password or PIN information, user device registrations, a second-level authentication, or a third-level authentication. In various embodiments, the users' identities may be verified by the HIE server 106. Information provided by the users in a real-time may be used, by the HIE server 106, to confirm the users' identities. In various embodiments, the users' identities may be verified using a name, a password, one or security questions, or a combination thereof. In various embodiments, a user may be identified using an encryption key and/or a decryption key.

In various embodiments, the data stored in the group of databases 102b may be accessed at different levels, for example using a first level subsystem and a second level subsystem. In various embodiments, a user may directly access the first level subsystem. To access data stored in the second level subsystem, the second level subsystem may be accessed through the first level subsystem. It should be noted that the communication between the first level subsystem and the second level subsystem may be encrypted. In an example, the second level subsystem may be implemented over a blockchain network (such as a PTOYNet blockchain network or a PTOYNet Ethereum™ Blockchain network). In various embodiments, the blockchain network may be used to implement smart contracts.

In accordance with various embodiments, a primary care physician may input data into the HIE server 106 using the user device 102. The data may be processed by the first level subsystem and the second level subsystem. This may be done successively. The data may be stored on the first level subsystem and/or the second level subsystem of the HIE server 106. This may be done successively. The data may include, but not limited to, one or more instructions to a patient to see a physician specialist. Further, the data may be stored in one or more blockchains of the second level subsystem. Successively, the patient may be able to access the data relating to the patient's care provided by the primary care physician. Successively, the patient may be able to retrieve the data using the user device 102 of the patient.

Thereafter, the patient may communicate with the physician specialist using the HIE server 106. It should be noted that the physician specialist may be able to access the data of the patient from the first level subsystem and/or the second level subsystem. Further, the physician specialist may be able to communicate with the patient. It should be noted that all (or substantially all) communications between the primary care physician, the physician specialist and the patient may be stored and may be accessible on a blockchain network (such as a PTOYNet blockchain network or a PTOYNet Ethereum™ Blockchain network).

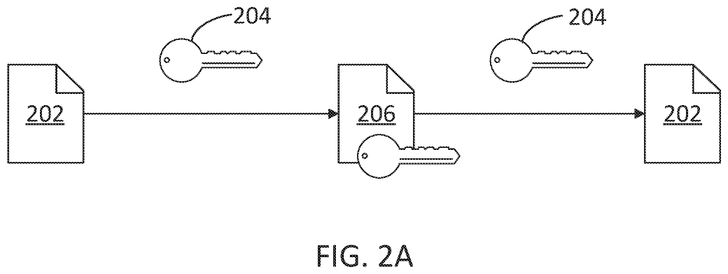

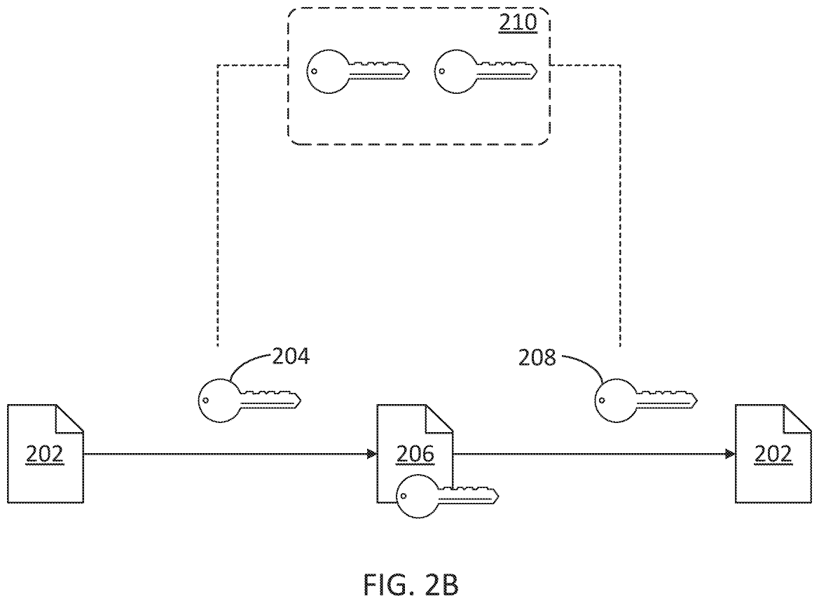

FIG. 2A illustrates a method for symmetric encryption of data, in accordance with various embodiments. Within the method, original data 202 may be encrypted using a key 204 to obtain an encrypted data 206. Thereafter, the encrypted data 206 may be decrypted using the key 204 to obtain back the original data 202. It should be noted that encryption and decryption of the data may be performed using a same key. Further, one or more parties involved in a communication may have the same key to encrypt and decrypt the data. In some embodiments, key 204 may be stored in a key database that is part of a blockchain database (e.g., key database 130b).

FIG. 2B illustrates a method for asymmetric encryption of data, in accordance with various embodiments. During the method, original data 202 may be encrypted using a key 204 to obtain encrypted data 206. Thereafter, the encrypted data 206 may be decrypted using another key 208 to obtain the original data 202. It should be noted that encryption and decryption of the data may be performed using different keys e.g., a key pair 210. In some embodiments, any one or more of keys 204 and 208, and key pair 210, may be stored in a key database that is part of a blockchain database (e. g., key database 130b).

In some embodiments, the steps illustrated in FIGS. 2A-B may be initiated by users who generate a new profile on the blockchain network. Private keys may be stored in decentralized and distributed hashes through the blockchain network. In some embodiments, the steps illustrated in FIGS. 2A-B may be partially performed in either one of devices 102 and 104, in HIE server 106, or in network server 108. For example, in some embodiments, HIE server 106 may install a software development kit (SDK) or a key generator application in devices 102 or 104 to perform at least some of the steps illustrated in FIGS. 2A-B. Likewise, keys 204, 208, and key pair 210 may be stored in a memory of either one of devices 102 and 104, in HIE server 106, or in network server 108, or in an associated database (e.g., any one of databases 102b).

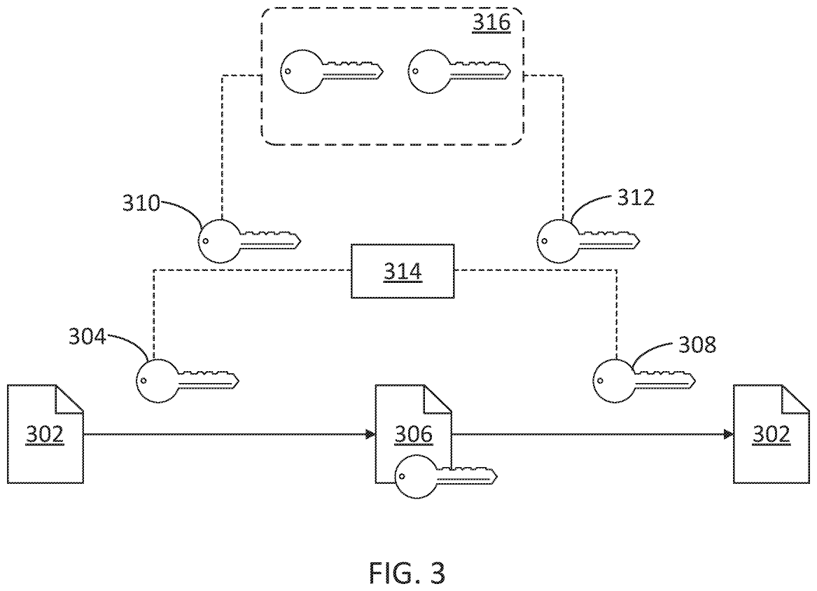

FIG. 3 illustrates a method for hybrid encryption of data, in accordance with various embodiments. During the method, both symmetric encryption and asymmetric encryption techniques may be used in tandem. In various embodiments, the symmetric encryption technique may be used to encrypt data 302 using a symmetric key 304 for producing encrypted data 306. The encrypted data 306 may be decrypted using another symmetric key 308 for obtaining back data 302. Further, a public key 310 may be used to encrypt the symmetric key 304 and a private key 312 may be used to encrypt the symmetric key 308, stored as an encrypted key 314. The public key 310 and the private key 312 may form a key pair 316. In some embodiments, any one or more of keys 304 and 308, public key 310, private key 312, and key pair 316 may be stored in a key database that is part of a blockchain database (e.g., key database 130b).

In some embodiments, the steps illustrated in FIG. 3 may be initiated by users who generate a new profile on the blockchain network. Private keys may be stored in decentralized and distributed hashes through the blockchain network. In some embodiments, the steps illustrated in FIG. 3 may be partially performed in either one of devices 102 and 104, in HIE server 106, or in network server 108. For example, in some embodiments, HIE server 106 may install a software development kit (SDK) or a key generator application in devices 102 or 104 to perform at least some of the steps illustrated in FIG. 3. Likewise, keys 204, 208, and key pair 210 may be stored in a memory of either one of devices 102 and 104, in HIE server 106, or in network server 108, or in an associated database (e.g., any one of databases 102b).

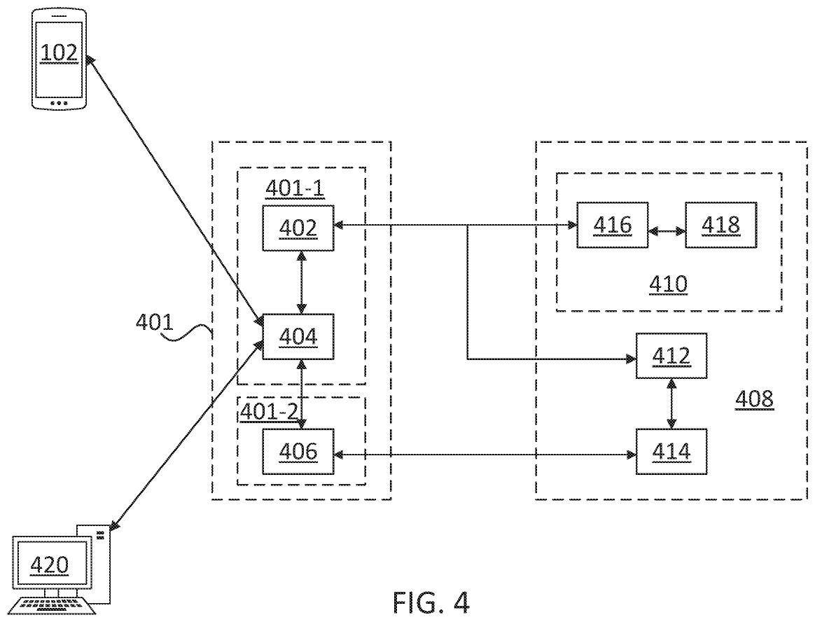

FIG. 4 illustrates a system 401 for storing and accessing data in a health care network, according to various embodiments. A first level subsystem 401-1 may include a core service component 402 and a Remote Procedure Call (RPC) component 404. A second level subsystem 401-2 may include a blockchain node 406. In various embodiments, first level subsystem 401-1 may include the core service component 402, and second level subsystem 401-2 may include the RPC component 404 and the blockchain node 406. To access blockchain node 406, user device 102 and a second user device 420 (e.g., a third party using a desktop) may place a remote call to RPC component 404. Blockchain node 406 may be a public node or a private node in a blockchain network having a layer over a public blockchain network, enabling the private node to perform private transactions via consensus algorithms (e.g., a Quorum blockchain node). Further, the core service component 402 of first level subsystem 401-1 may be present in communication with third-party servers and databases of a hospital computing network 408. The hospital computing network 408 may include a file system module 410, an EHR synchronization service 412, and a blockchain node 414 (e.g., a Quorum blockchain node). Further, the file system module 410 may include a file system manager 416 and a file system node 418. The blockchain node 406 of second level subsystem 401-2 may communicate with the blockchain node 414 of the hospital computing network 408. Patients may access the health care network for storing data through the user device 102, and a representative of a hospital may access the health care network through another user device 420.

In accordance with various embodiments, the representative of the hospital may want to synchronize Electronic Health Record (EHR) data of a patient, e.g., by using corresponding blockchain hashes. Successively, first level subsystem 401-1 and second level subsystem 401-2 may ask the patient for permission to allow a representative of the hospital to store the EHR data of the patient, through the file system module 410. Based at least on the permission granted by the patient, a signed transaction may be created to confirm the permission of the hospital to store the EHR data. Further, the signed transaction may activate a smart contract that may add hospital identification information such as a blockchain address to a list of permitted users. In some embodiments, the signed transaction and the smart contract are stored in file system module 410.

Further, the signed transaction may be transmitted from the user device 102 to the RPC component 404 of the first level subsystem and/or the second level subsystem. The RPC component 404 may communicate the signed transaction to the blockchain node 406 of the second level subsystem. This may be done successively. The blockchain node 406 may activate one or more smart contracts. This may be done successively. Thereafter, the blockchain node 406 may revise a state of one or more blockchains.

Further, based at least on the permission granted by the patient, the EHR synchronization service may obtain a list of patients from the RPC component 404. Further, the EHR synchronization service may confirm whether the patient has granted permission. Based at least on the permission, the first level subsystem and the second level subsystem may obtain the EHR data and may calculate a hash function for the EHR data. The HIE server 106 may match the hash function of the EHR data with a hash function for the patient blockchain on the blockchain node 406 of the second level subsystem. This may be done successively. Thereafter, if the hash function of the EHR data matches with the hash function for the patient blockchain on the blockchain node 406 of the second level subsystem, the EHR data of the patient may remain unchanged.

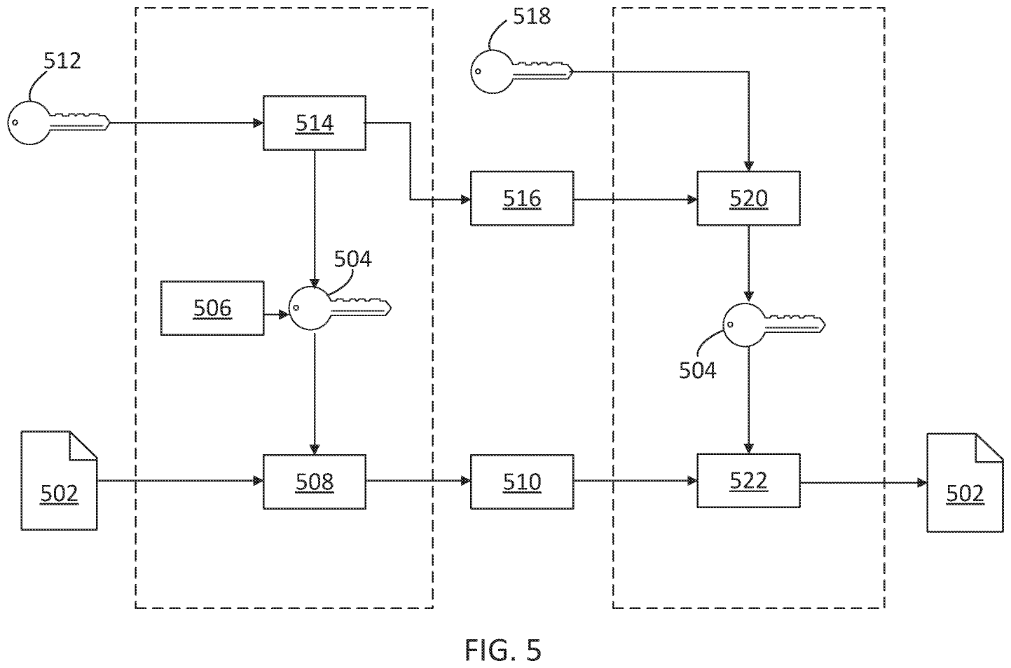

FIG. 5 illustrates a system for storing and accessing data in a health care network implemented, for example, over a blockchain network, according to various embodiments (cf. FIGS. 1 and 4). In some embodiments, the HIE server 106 may execute an application for determining permission from the user for obtaining EHR data 502. In various embodiments, if the user grants the permission, the HIE server 106 may obtain the EHR data 502 for calculating a hash function for the EHR data 502. Further, the HIE server 106 may match the hash function of the EHR data 502 with a hash function for the user blockchain on the blockchain node of the second level sub-system. In various embodiments, if the two hash matches, there is no change to the user's EHR data 502. In various embodiments, the two hash functions do not match, the HIE server 106 may generate a random string e,g, secret key 504, through a random key generator 506. Accordingly, in some embodiments secret key 504 may be a random string. The secret key 504 may be used for Advanced Encryption Standard (AES) encryption of the EHR data 502, in an AES encryptor 508, for generating encrypted EHR data 510.

In accordance with various embodiments, the secret key 504 may then be encrypted by a Rivest-Shamir-Adleman (RSA) public key 512 of the patient, in an RSA encryptor 514, to generate an encrypted secret key 516. The HIE server 106 may further send the encrypted EHR data 510 to the core service component 402 for forwarding the data to the file system manager 416 of the hospital computing network 408 for storage. Further, the file system manager 416 may send a file system hash function to the core service component 402 for further sending the Inter Planetary File System (IPFS) hash function to EHR synchronization service 412. The EHR synchronization service 412 may further update the patient smart contract with the new file system hash function, the encrypted random key, a hash function of the unencrypted file, and file name.

In accordance with various embodiments, a hospital representative, such as a doctor or a hospital administration, may want to view the EHR data 502. In such a scenario, the user may first send a signed transaction to a RPC component 404 for granting permission to the hospital representative to view the EHR data 502. Once the permission is granted, the signed transaction may be added to the blockchain node 414 and a new smart contract will be created for a blockchain corresponding to the hospital representative. After adding the signed transaction, the hospital representative may be able to view the EHR data 502 of the user, on a device.

In accordance with various embodiments, in order to view the EHR data 502 on the device, the HIE server 106 may collect the encrypted EHR data 510 from the user's blockchain and may decrypt the encrypted EHR data 510 using patient's RSA private key 518. The HIE server 106 may decrypt the encrypted secret key 516, in an RSA decryptor 520, using RSA private key of the hospital representative. The encrypted EHR data 510 may be decrypted using the RSA public key 512 of the hospital representative, in an AES decryptor 522. This may be done successively. Further, the HIE server 106 may load the decrypted EHR data 502 to the smart contract previously created for the hospital representative.

Post loading, the RPC component 404 may obtain the signed transaction from the patient's user device and transmit the signed transaction to the blockchain node 406 of the second level subsystem. The blockchain node 406 may confirm ownership of the signed transaction and may execute the smart contract for the hospital representative to view the user's health information. This may be done successively.

In accordance with various embodiments, the patient may decline permission for the hospital representative to access the EHR data 502. In such a scenario, the user, through a user device, may send a signed transaction revoking permission to the RPC component 404. The RPC component 404 may forward the signed transaction to the blockchain node 406 of the second level subsystem. This may be done successively. The blockchain node 406 may confirm ownership of the signed transaction and may delete the smart contract previously created to allow the hospital representative to have access to the patient's EHR data 502. This may be done successively.

Further, the HIE server 106 may include a health record network for an intermediary enabling sharing of user's medical records with providers. For enabling sharing, the user may grant specific permissions to the providers for accessing parts of the user's medical records stored in a user database (not shown) implemented over a blockchain network. The user may also grant specific permissions to modify the user's medical records in the user database. In various embodiments, the user may include of any users constituting a value chain, such as doctors, nurses etc. In various embodiments, the user may be remote doctors logging into the HIE server 106 or doctors present in hospitals.

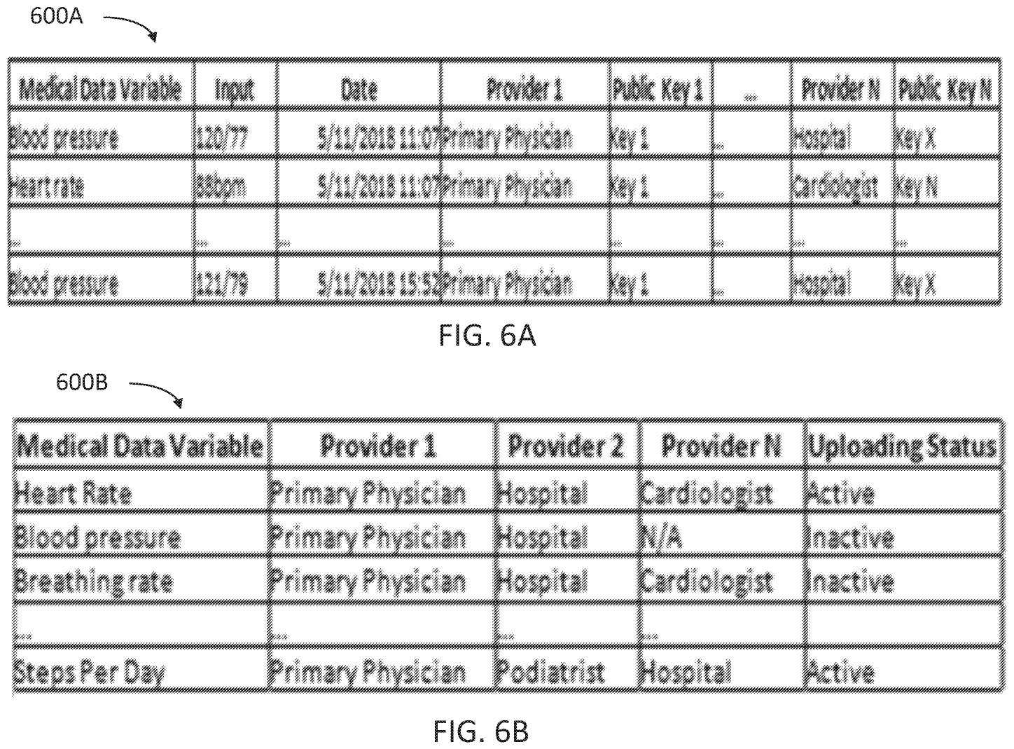

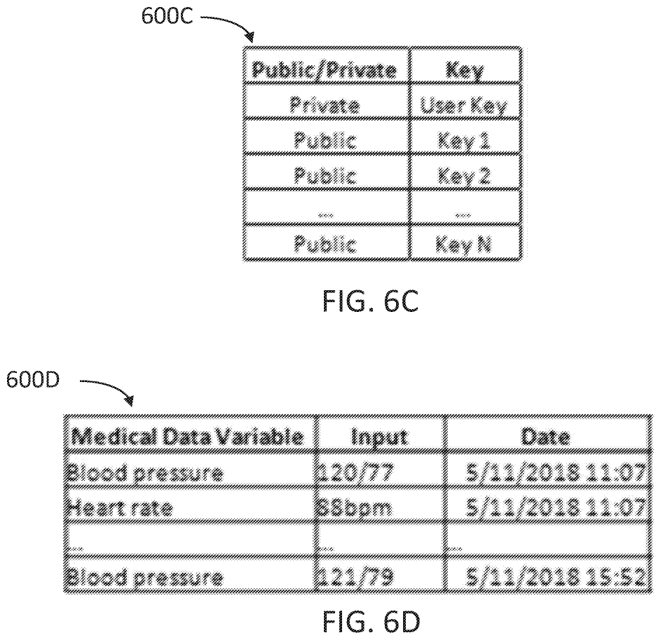

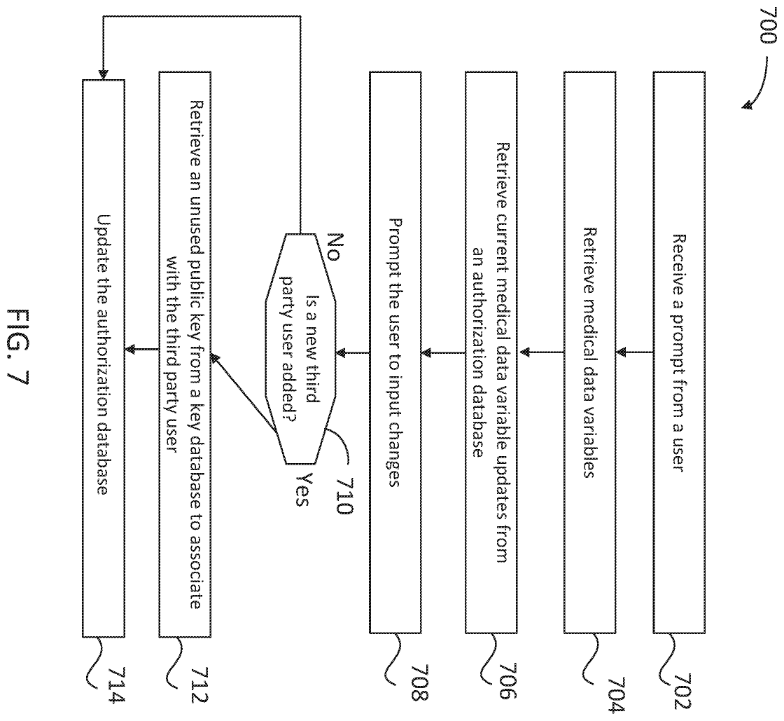

FIG. 6A illustrates an example of a table 600A showing various example types of information stored in a patient blockchain database, according to various embodiments. In accordance with various embodiments, the user device 102 may be connected with the blockchain network server 106, through the communication network 110. The blockchain network server 106 may include a patient blockchain database 134 and a key generator module 136. In various embodiments, the patient blockchain database 134 may be configured to store a user's medical data encrypted in a blockchain. In some embodiments, the user's medical data may include, but not limited to, medical data variable, an input, date, provider's name, or a public key. It should be noted that the patient blockchain database 134 may be accessed by authorized third parties. Further, the user device 102 may include an application programming interface (API) 132a for allowing the user to access or to view the medical data.

FIG. 6B illustrates an example of a table 600B showing various example types of information stored in an authorization database (e.g., authorization database 128b), according to various embodiments. In accordance with various embodiments, the authorization database 128b may be configured to store information related to medical data. For example, the information may include, but is not limited to, medical data variables, one or more provider names, and uploading status. In various embodiments, the uploading status may be active or inactive. The authorization database 128b may store names of the providers that are accessing the medical data. Further, the authorization database 128b may store information about the medical data variables that are being uploaded to the patient blockchain database 134. It should be noted that the information related to the medical data variables accessed by a user, may be received from the medical selection module 124. Further, the uploading status may be received from the system control module 124.

FIG. 6C illustrates an example of a table 600C showing various example types of information stored in a key database (e.g., key database 130b), according to various embodiments. In accordance with various embodiments, the key database 130b may be configured to store keys. For example, the keys may be public keys, private keys or user's key. Further, the public keys may be generated by the key generator module 136 of the HIE server 106. Further, the private keys and multiple public keys may be accessed by third party users to access the authorized data.

FIG. 6D illustrates an example of a table 600D showing various types of information stored in medical records database (e.g., medical records database 132b), according to various embodiments. In accordance with various embodiments, the medical records database 132b may be configured to store user's medical data. The user's medical data may be received from the wearable device 104. The medical records database 132b may be present in a medical application running on the user device 102. For example, the medical records database 132b may include medical data variable, an input data, a date and a time stamp. Further, the medical records database 132b may be used by the monitor and report module 128a to encrypt data and to store the data securely in the patient blockchain database 134.

FIG. 7 illustrates an example of a flowchart 700 showing a method performed by the medical selection module 124. Functioning of the medical selection module 132 will now be explained with reference to flowchart 700. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In accordance with various embodiments, the medical selection module 124 may receive a prompt from a user, at step 702. The medical selection module 124 may retrieve medical data variables, at step 704. This may be done successively. It should be noted that information related to the medical data variables may be retrieved from the wearable device 104. In various embodiments, the wearable device 104 may include information such as heart rate, blood pressure, or a number of steps moved per day. The medical selection module 124 may retrieve current medical data variables updates from the authorization database 128b, at step 706. This may be done successively. The current medical data variables updates may include, for example, information related to a name of a third party user accessing the medical data variables.

The medical selection module 124 may prompt the user to input changes, at step 708. This may be done successively. The user may be prompted to input changes for what medical data variables collected by the wearable device 104 and who is accessing the medical data variables. In various embodiments, the medical selection module 124 may determine what medical data variables are being accessed by providers, health networks, and other third parties. The medical selection module 124 may determine whether a new third party user is added, at step 710. This may be done successively. The new third party user may be a doctor such as a cardiologist. In various embodiments, if the new third party user is added, then the medical selection module 124 may retrieve an unused public key from the key database 130b to associate with the third party user, at step 712. The medical selection module 124 may update the authorization database 128b, at step 714. This may be done successively. In various embodiments, if the new third party user is not added, then the medical selection module 124 may follow the step 714.

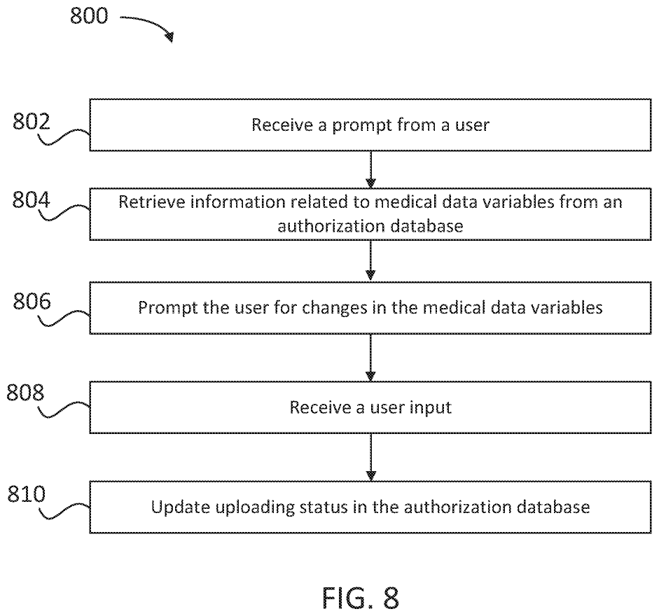

FIG. 8 illustrates an example of a flowchart 800 showing a method performed by the system control module 124, according to various embodiments. Functioning of the system control module 124 will now be explained with reference to the flowchart 800 shown in FIG. 8. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In accordance with various embodiments, the system control module 124 may receive a prompt from the user, at step 802. In various embodiments, the prompt may indicate that the user wants to start or stop an input of the medical data variables into a blockchain. The system control module 124 may retrieve information related to the medical data variables from the authorization database 128b, at step 804. This may be done successively. The information may be uploading status of the medical data variables. In various embodiments, the uploading status may be active or inactive. The system control module 124 may prompt the user for changes in the medical data variables, at step 806. This may be done successively. In an example, the user may be prompted to input changes pertaining to an active status of the medical data variables. The system control module 124 may receive a user input, at step 808. This may be done successively. Thereafter, the system control module may update the uploading status in the authorization database 128b, at step 810. In various embodiments, when the user updates the uploading status, the system control module 124 may allow the input changes of the medical data variable to be temporarily activated or deactivated for allowing the user to have control over what data is being sent to the blockchain.

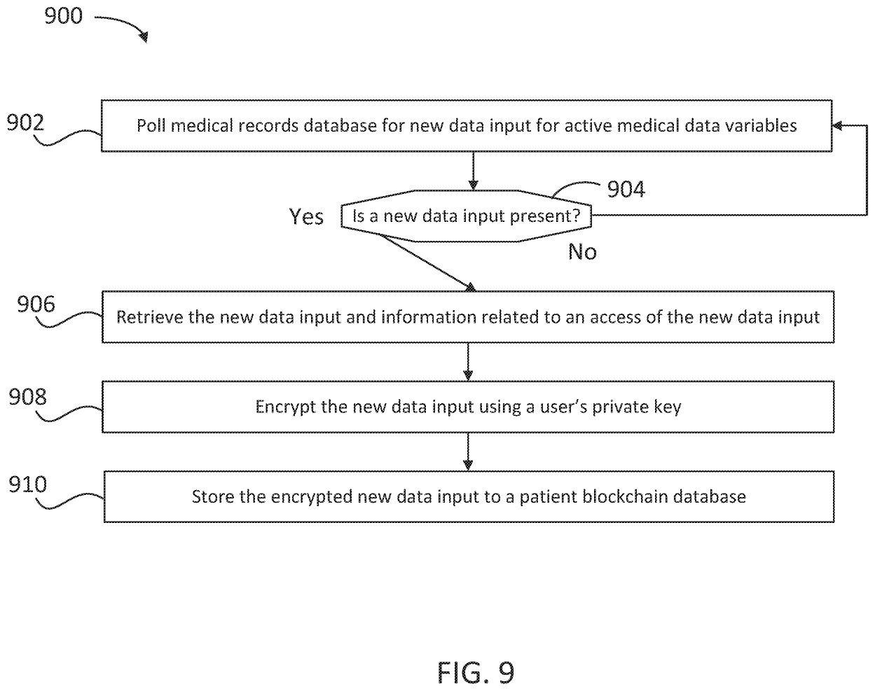

FIG. 9 illustrates an example of a flowchart 900 showing a method performed by the monitor and report module 128a, in accordance with various embodiments. Functioning of the monitor and report module 128a will now be explained with reference to the flowchart 900 shown in FIG. 9. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In various embodiments, the monitor and report module 128a may poll the medical records database 132b for new data input for active medical data variables, at step 902. The monitor and report module 128a may determine whether a new data input is present, at step 904. This may be done successively. In various embodiments, if the new data input is not present, the monitor and report module 128a may follow the step 902. In various embodiments, if the new data input is present, then the monitor and report module 128a may retrieve the new data input and information related to an access of the new data input, at step 906. The information may be related to an individual who is accessing the new data input. The monitor and report module 128a may encrypt the new data input using a user's private key, at step 908. This may be done successively. The monitor and report module 128a may store the encrypted new data input to the patient blockchain database 134 of the HIE server 106, at step 910. This may be done successively.

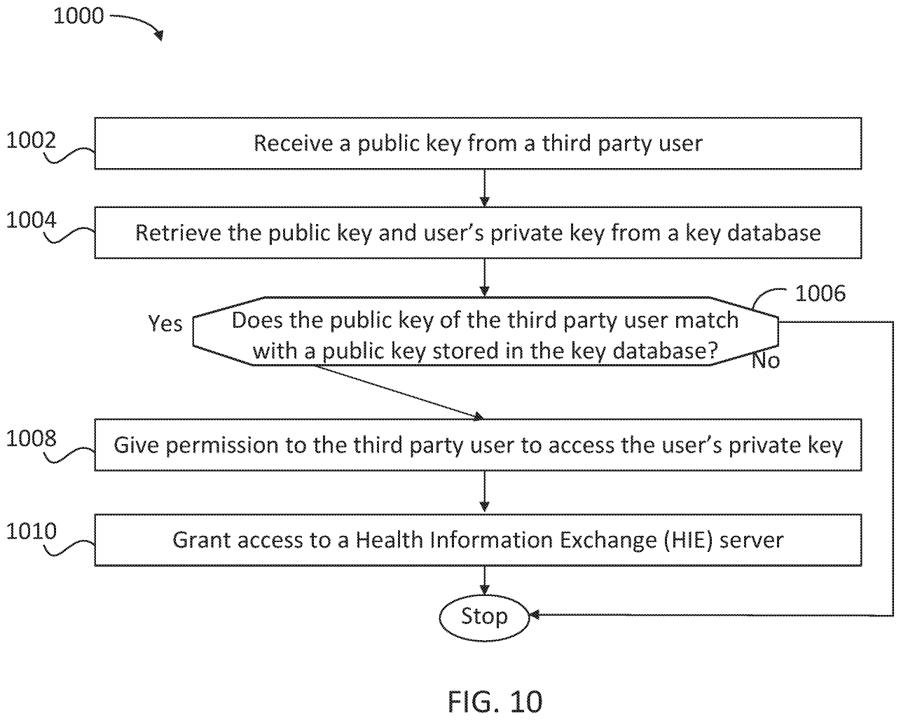

FIG. 10 illustrates an example of a flowchart 1000 showing a method performed by the secure module 130a, in accordance with various embodiments. Functioning of the secure module 130a will now be explained with reference to the flowchart 1000 shown in FIG. 10. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In accordance with various embodiments, the secure module 130a may receive a public key from the third party user, at step 1002. The third party user may refer to, for example, an individual belonging to hospitals, insurance companies, Contract Research Organizations (CROs), and drug companies. It should be noted that the public key may be assigned to the secure module 130a. The secure module 130a may retrieve the public key and user's private key from the key database 130b, at step 1004. This may be done successively. The secure module 130a may determine whether the public key of the third party user matches with a public key stored in the key database 130b, at step 1006. This may be done successively. In various embodiments, if the public key of the third party user matches with the public key stored in the key database 130b, then the secure module 130a may give permission to the third party user to access the user's private key, at step 1008. Thereafter, the secure module 130a may grant access to the HIE server 106, at step 1010. It should be noted that the access may be granted to retrieve authorized information. In various embodiments, if the public key of the third party user does not match the public key stored in the key database 130b, then the secure module 130a may end the process.

In accordance with various embodiments, the wearable device 104 may collect user's health information, for example, on May 11, 2018 at 11:07 A.M. At first, user's blood pressure may be 120/77 and user's heart rate may be 88 beats per minute. Few hours later, for example, the wearable device 104 may again collect the user's health information such as the user's blood pressure being 121/79. The user's health information may be stored in the medical records database 132b, through the user device 102. The user device 102 may include of multiple modules and databases. The medical selection module 124 may be activated when the user prompts by opening an application or selecting an option through the API 132a. The module selection module 122 may retrieve the medical variables being collected by the wearable device 104 from the medical records database 132b. The medical variables may include, for example, user's blood pressure, heart rate, or a number of steps moved per day.

In accordance with various embodiments, the medical selection module 124 may retrieve current medical updates from the authorized database 128 including providers showing user's primary physician and hospital having access to the medical data variables. In various embodiments, a cardiologist may have an access to the heart rate and breathing rate, and a podiatrist may have an access to the number of steps moved per day data. In various embodiments, the medical selection module 124 may prompt the user to input changes, such as adding the third party user to the medical data variables, removing the third party user from a medical data variable information, adding a new medical data variable to be tracked, or removing the medical data variable.

In accordance with various embodiments, if the third party user is added, then a public key may be retrieved from the key database 130b to send and associate with the third party user. Further, the third party user may use the public key to access the authorized data. Once the public key is obtained, the authorization database 128b may update the information regarding the user's health. In various embodiments, the authorization database 128b may also be updated if the new third party user is not added to the medical data variables.

After updating the authorization database 128b, the system control module 124 may be activated when receives a prompt from the user. The prompt may correspond to changing the uploading status of the medical data variables to the HIE server 106. The system control module 124 may retrieve an active status of the medical data variables from the authorization database 128b, where the heart rate and the number of steps moved per day are active, and the blood pressure and the breathing rating are inactive. Further, the system control module 124 may prompt the user to make changes to the uploading status of the medical data variables. In various embodiments, the user may change the blood pressure status to active. Thereafter, the system control module 124 may receive the user's input and may update the upload status in the authorization database 128b.

The monitor and report module 128a may poll the medical records database 132b for the new data input. This may be done successively. If there is a new data input such as a new heart rate input and a new blood pressure input, then the monitor and report module 128a may retrieve the new data input from the medical records database 132b for acknowledging who can access the new data input from the authorization database 128b. Further, the new data input may be encrypted using the user's private key from the key database 130b and then uploaded to the patient blockchain database 134 of the HIE server 106.

Further, the secure module 130a may be activated when the third party user accesses the user's health information in the patient blockchain database 134. The secure module 130a may retrieve the private keys and the public keys from the key database 130b on the user device 102. Further, the secure module 130a may verify if the public key of the third party user is present in the key database 130b. Thereafter, the secure module 130a may provide permission to the third party user to access the user's private key and to access the authorized data in the patient blockchain database 134 of the HIE server 106. In various embodiments, for example, the user's blood pressure input is 120/77 at 11:07 on May 11, 2018 and a primary physician has access using Key 1 and the hospital has access using Key X.

### Computer System

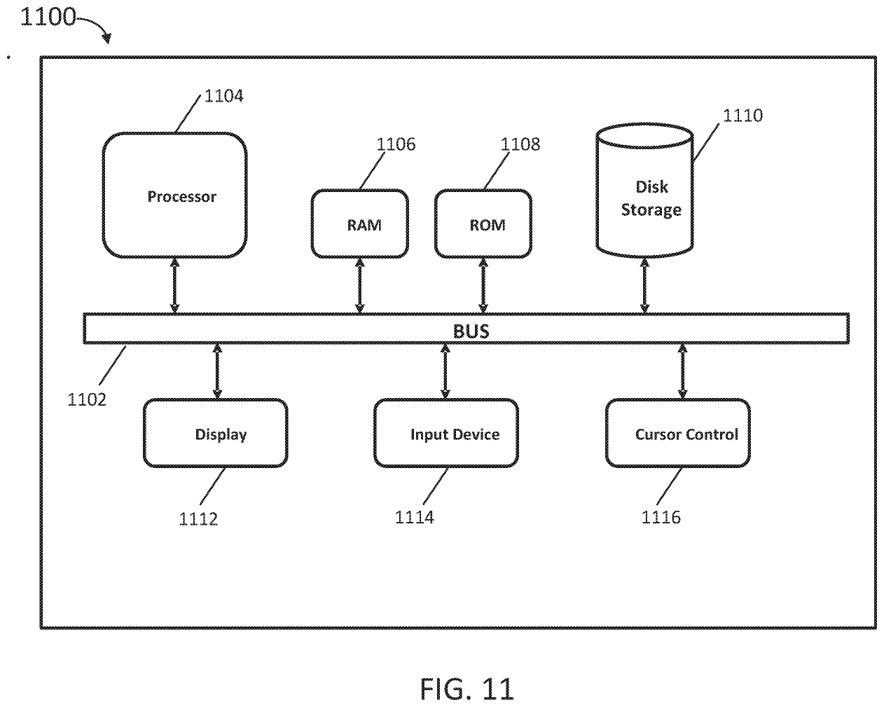

FIG. 11 is a block diagram that illustrates a computer system 1100, upon which embodiments, or portions of the embodiments, of the present teachings may be implemented. In various embodiments of the present teachings, computer system 1100 can include a bus 1102 or other communication mechanism for communicating information, and a processor 1104 coupled with bus 1102 for processing information. In various embodiments, computer system 1100 can also include a memory 1106, which can be a random access memory (RAM) or other dynamic storage device, coupled to bus 1102 for determining instructions to be executed by processor 1104. Memory 1106 also can be used for storing temporary variables or other intermediate information during execution of instructions to be executed by processor 1104. In various embodiments, computer system 1100 can further include a read-only memory (ROM) 1108 or other static storage device coupled to bus 1102 for storing static information and instructions for processor 1104. A storage device 1110, such as a magnetic disk or optical disk, can be provided and coupled to bus 1102 for storing information and instructions.

In various embodiments, computer system 1100 can be coupled via bus 1102 to a display 1112, such as a cathode ray tube (CRT) or liquid crystal display (LCD), for displaying information to a computer user. An input device 1114, including alphanumeric and other keys, can be coupled to bus 1102 for communicating information and command selections to processor 1104. Another type of user input device is a cursor control 1116, such as a mouse, a trackball or cursor direction keys for communicating direction information and command selections to processor 1104 and for controlling cursor movement on display 1112. This input device 1114 typically has two degrees of freedom in two axes, a first axis (e.g., x) and a second axis (e.g., y), that allows the device to specify positions in a plane. However, it should be understood that input devices 1114 allowing for 3-dimensional (x, y, and z) cursor movement are also contemplated herein.

Consistent with certain implementations of the present teachings, results can be provided by computer system 1100 in response to processor 1104 executing one or more sequences of one or more instructions contained in memory 1106. Such instructions can be read into memory 1106 from another computer-readable medium or computer-readable storage medium, such as storage device 1110. Execution of the sequences of instructions contained in memory 1106 can cause processor 1104 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 1104 for execution. Such a medium can take many forms, including but not limited to, non-volatile media, volatile media, and transmission media. Examples of non-volatile media can include, but are not limited to, optical, solid state, and magnetic disks, such as storage device 1110. Examples of volatile media can include, but are not limited to, dynamic memory, such as memory 1106. Examples of transmission media can include, but are not limited to, coaxial cables, copper wire, and fiber optics, including the wires that include bus 1102.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to a computer-readable medium, instructions or data can be provided as signals on transmission media included in a communications apparatus or system to provide sequences of one or more instructions to processor 1104 of computer system 1100 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein including flow charts, diagrams, and accompanying disclosure can be implemented using computer system 1100 as a standalone device or on a distributed network of shared computer processing resources such as a cloud computing network.

In accordance with various embodiments, the systems and methods described herein can be implemented using computer system 1100 as a standalone device or on a distributed network of shared computer processing resources such as a cloud computing network. As such, a non-transitory computer-readable medium can be provided in which a program is stored for causing a computer to perform the disclosed methods for identifying mutually incompatible gene pairs.

It should also be understood that the preceding embodiments can be provided, in whole or in part, as a system of components integrated to perform the methods described. For example, in accordance with various embodiments, the methods described herein can be provided as a system of components or stations for analytically determining novelty responses.

In describing the various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments. Similarly, any of the various system embodiments may have been presented as a group of particular components. However, these systems should not be limited to the particular set of components, their specific configuration, communication, and physical orientation with respect to each other. One skilled in the art should readily appreciate that these components can have various configurations and physical orientations (e.g., wholly separate components, units, and subunits of groups of components, different communication regimes between components).

Embodiments disclosed herein include:

A. A computer-implemented method for accessing a patient's health information includes configuring a health information exchange server in a health care network that includes a blockchain network to communicate with a user device present in communication with the health information exchange server. The computer-implemented method also includes providing an access to the patient's health information to a third party user based on a patient's permission, wherein the patient's health information includes a blockchain string in the blockchain network, and providing the access to the third party user includes providing an encrypted key to decode the blockchain string.

B. A system for accessing a patient's health information includes a memory storing instructions and one or more processors configured to execute the instructions. The instructions cause the system to communicate with a health information exchange server in a health care network that includes a blockchain network and to provide an access to the patient's health information to a third party user based on a patient's permission, wherein the patient's health information includes a blockchain string in the blockchain network, and providing the access to the third party user includes providing an encrypted key to decode the blockchain string. The instructions also cause the system to update the patient's health information using a wearable device worn by a user, wherein the wearable device is present in communication with a user device that has access to the health care network.

C. A computer-implemented method for accessing a patient's health information includes communicating, from a server in a healthcare network that includes a blockchain network, with a user having a user device, through the healthcare network. The computer-implemented method also includes prompting the user to update the patient's health information with the user device, and receiving a request from a third party user to access the patient's health information. The computer-implemented method also includes providing an access to the patient's health information to the third party user based on a patient's permission, wherein the patient's health information includes a blockchain string in the blockchain network, and providing the access to the third party user includes providing an encrypted key to decode the blockchain string.

Each of embodiments A, B and C may have one or more of the following additional elements in any combination: Element 1, further including updating the patient's health information using a wearable device worn by a user, wherein the wearable device is present in communication with the user device. Element 2, wherein the patient's health information includes blood pressure, heart rate, and number of steps moved per day, further including uploading the blood pressure, heart rate, and number of steps moved per day as a new block in the blockchain string. Element 3, wherein the third party user refers to an individual belonging to hospitals, insurance companies, Contract Research Organizations (CROs), and drug companies, further including providing an access to the patient's health information based on a hospital, insurance company, or CRO where the third party user belongs. Element 4, wherein the encrypted key is a public key, and providing an access to the patient's health information to a third party user includes determining whether the third party user already exists in a database, and providing an unused public key from a key database to the third party user when the third party user is new to the database. Element 5, wherein providing an access to the patient's health information to a third party user includes receiving, from the third party user, a public key and providing the encrypted key in response to identifying the public key in a key database, wherein the encrypted key is a private key matching the public key in the key database. Element 6, further including polling, with the user device, multiple medical records in a medical records database through a communication network, and retrieving a new data input from the medical records, and a metadata associated with the new data input. Element 7, further including retrieving a new data input from a medical records database, encrypting the new data input with a private key, adding the new data input to a new block in the blockchain string, and storing the blockchain string in a patient blockchain in the blockchain network. Element 8, further including activating the user device when the third party user accesses the patient's health information in the blockchain string through the blockchain network, and authorizing the third party user to access the patient's health information when a public key with the third party user is in a key database. Element 9, further including providing a new data input to a medical records database in response to a prompt by the health information exchange server, wherein the new data input includes a new value of a medical data variable.

Each of embodiments A, B and C may also have one or more of the following additional elements in any combination: Element 10, wherein the one or more processors execute instructions to upload the blood pressure, heart rate, and number of steps moved per day as a new block in the blockchain string. Element 11, wherein the one or more processors execute instructions to provide an access to the patient's health information based on a hospital, insurance company, or CRO where the third party user belongs. Element 12, wherein the one or more processors execute instructions to determine whether the third party user already exists in a database, and to provide an unused public key from a key database to the third party user when the third party user is new to the database. Element 13, wherein the one or more processors execute instructions to receive, from the third party user, a public key and to provide the encrypted key in response to identifying the public key in a key database, wherein the encrypted key is a private key matching the public key in the key database.

Each of embodiments A, B and C may also have one or more of the following additional elements in any combination: Element 14, further including updating the patient's health information with a new block in the blockchain string. Element 15, wherein the encrypted key is a public key, and providing an access to the patient's health information to a third party user includes determining whether the third party user already exists in a database, and providing an unused public key from a key database to the third party user when the third party user is new to the database. Element 16, wherein providing an access to the patient's health information to a third party user includes receiving, from the third party user, a public key and providing the encrypted key in response to identifying the public key in a key database, wherein the encrypted key is a private key matching the public key in the key database. Element 17, wherein the user device includes an image-capturing device, and prompting the user to update the patient's health information includes capturing an image of a medical device indicating a medical data variable.

It will be appreciated that variants of the above disclosed, and other features and functions or alternatives thereof, may be combined into many other different systems or applications. Presently unforeseen or unanticipated alternatives, modifications, variations, or improvements therein may be subsequently made by those skilled in the art that are also intended to be encompassed by the following claims.

What is claimed is:

1. A computer-implemented method for accessing a patient's health information, the method comprising:
configuring a health information exchange server in a health care network that includes a blockchain network to communicate with a user device present in communication with the health information exchange server; and
providing an access to the patient's health information to a third party user based on a patient's permission, wherein the patient's health information includes a blockchain string in the blockchain network, and providing the access to the third party user comprises providing an encrypted key to decode the blockchain string.

2. The computer-implemented method of claim 1, further comprising updating the patient's health information using a wearable device worn by a user, wherein the wearable device is present in communication with the user device.

3. The computer-implemented method of any one of claims 1 and 2, wherein the patient's health information comprises blood pressure, heart rate, and number of steps moved per day, further comprising uploading the blood pressure, heart rate, and number of steps moved per day as a new block in the blockchain string.

4. The computer-implemented method of any one of claims 1 through 3, wherein the third party user refers to an individual belonging to hospitals, insurance companies, Contract Research Organizations (CROs), and drug companies, further comprising providing an access to the patient's health information based on a hospital, insurance company, or CRO where the third party user belongs.

5. The computer-implemented method of any one of claims 1 through 4, wherein the encrypted key is a public key, and providing an access to the patient's health information to a third party user comprises determining whether the third party user already exists in a database, and providing an unused public key from a key database to the third party user when the third party user is new to the database.

6. The computer-implemented method of any one of claims 1 through 5, wherein providing an access to the patient's health information to a third party user comprises receiving, from the third party user, a public key and providing the encrypted key in response to identifying the public key in a key database, wherein the encrypted key is a private key matching the public key in the key database.

7. The computer-implemented method of any one of claims 1 through 6, further comprising polling, with the user device, multiple medical records in a medical records database through a communication network, and retrieving a new data input from the medical records, and a metadata associated with the new data input.

8. The computer-implemented method of any one of claims 1 through 7, further comprising retrieving a new data input from a medical records database, encrypting the new data input with a private key, adding the new data input to a new block in the blockchain string, and storing the blockchain string in a patient blockchain in the blockchain network.

9. The computer-implemented method of any one of claims 1 through 8, further comprising activating the user device when the third party user accesses the patient's health information in the blockchain string through the blockchain network, and authorizing the third party user to access the patient's health information when a public key with the third party user is in a key database.

10. The computer-implemented method of any one of claims 1 through 9, further comprising providing a new data input to a medical records database in response to a prompt by the health information exchange server, wherein the new data input includes a new value of a medical data variable.

11. A system for accessing a patient's health information, the system comprising:
a memory storing instructions; and
one or more processors configured to execute the instructions and cause the system to:
communicate with a health information exchange server in a health care network that includes a blockchain network;
provide an access to the patient's health information to a third party user based on a patient's permission, wherein the patient's health information includes a blockchain string in the blockchain network, and providing the access to the third party user comprises providing an encrypted key to decode the blockchain string; and
update the patient's health information using a wearable device worn by a user, wherein the wearable device is present in communication with a user device that has access to the health care network.

12. The system of claim 11, wherein the patient's health information comprises blood pressure, heart rate, and number of steps moved per day, and the one or more processors execute instructions to upload the blood pressure, heart rate, and number of steps moved per day as a new block in the blockchain string.

13. The system of any one of claims 11 and 12, wherein the third party user refers to an individual belonging to hospitals, insurance companies, Contract Research Organizations (CROs), and drug companies, and the one or more processors execute instructions to provide an access to the patient's health information based on a hospital, insurance company, or CRO where the third party user belongs.

14. The system of any one of claims 11 through 13, wherein the encrypted key is a public key, and to provide an access to the patient's health information to a third party user the one or more processors execute instructions to determine whether the third party user already exists in a database, and to provide an unused public key from a key database to the third party user when the third party user is new to the database.

15. The system of any one of claims 11 through 14, wherein to provide an access to the patient's health information to a third party user the one or more processors execute instructions to receive, from the third party user, a public key and to provide the encrypted key in response to identifying the public key in a key database, wherein the encrypted key is a private key matching the public key in the key database.

16. A computer-implemented method for accessing a patient's health information, comprising:
communicating, from a server in a healthcare network that includes a blockchain network, with a user having a user device, through the healthcare network;
prompting the user to update the patient's health information with the user device;
receiving a request from a third party user to access the patient's health information; and
providing an access to the patient's health information to the third party user based on a patient's permission, wherein the patient's health information includes a blockchain string in the blockchain network, and providing the access to the third party user comprises providing an encrypted key to decode the blockchain string.

17. The computer-implemented method of claim 16, further comprising updating the patient's health information with a new block in the blockchain string.

18. The computer-implemented method of any one of claims 16 and 17, wherein the encrypted key is a public key, and providing an access to the patient's health information to a third party user comprises determining whether the third party user already exists in a database, and providing an unused public key from a key database to the third party user when the third party user is new to the database.

19. The computer-implemented method of any one of claims 16 through 18, wherein providing an access to the patient's health information to a third party user comprises receiving, from the third party user, a public key and providing the encrypted key in response to identifying the public key in a key database, wherein the encrypted key is a private key matching the public key in the key database.

20. The computer-implemented method of any one of claims 16 through 19, wherein the user device includes an image-capturing device, and prompting the user to update the patient's health information comprises capturing an image of a medical device indicating a medical data variable.
